(19) United States
(12) Patent Application Publication
(10) Pub. No.: US 2017/0186567 A1
(43) Pub. Date: Jun. 29, 2017

(54) OPERATION INPUT UNIT AND ENERGY TREATMENT INSTRUMENT

(71) Applicant: OLYMPUS CORPORATION, Tokyo (JP)

(72) Inventors: Hideo SANAI, Hachioji-shi (JP); Masami OSHIDA, Sagamihara-shi (JP)

(73) Assignee: OLYMPUS CORPORATION, Tokyo (JP)

(21) Appl. No.: 15/458,255

(22) Filed: Mar. 14, 2017

Related U.S. Application Data
(63) Continuation of application No. PCT/JP2015/084546, filed on Dec. 9, 2015.

(30) Foreign Application Priority Data
Dec. 19, 2014 (JP) 2014-257662

(51) Int. Cl. H01H 13/10; H01H 13/14; A61B 18/00

(52) U.S. Cl. CPC H01H 13/10; A61B 18/00; H01H 13/14; A61B 2018/00916

(57) ABSTRACT

An operation input unit includes a board unit including a switch, and a base. An external force application portion moves by a button portion being pushed, and changes an open or closed state of the switch by varying an external force acting on the board unit in accordance with the movement. A board deflection portion forms a space between the board deflection portion and the base in a state that the button portion is not pushed, and deflects toward a movement direction of the external force application portion by the external force application portion moving by the pushing of the button portion.

### CROSS-REFERENCE TO RELATED APPLICATIONS

This is a Continuation application of PCT Application No. PCT/JP2015/084546, filed Dec. 9, 2015 and based upon and claiming the benefit of priority from prior Japanese Patent Application No. 2014-257662, filed Dec. 19, 2014, the entire contents of which are incorporated herein by reference.

### BACKGROUND OF THE INVENTION

1. Field of the Invention

The present invention relates to an operation input unit including a board unit that is provided with a switch, and an energy treatment instrument including the operation input unit.

2. Description of the Related Art

U.S. Patent Application Publication No. 2005/0113824 discloses an energy treatment system including a switch board (board unit) which is provided with a switch. In an operation input unit provided in an energy treatment instrument of this energy treatment system, a button portion is pushed by an operation input, and thereby an external force application portion (pusher) moves, and external force acts on the switch from the external force application portion. Thereby, a movable contact portion and a fixed contact portion come in contact in the switch, and the switch enters a closed state (electrical conduction is established in the switch). By the electrical conduction in the switch being detected, high-frequency electric power is supplied to a treatment portion, and a high-frequency current flows through a treated target, such as a biological tissue, which is in contact with the treatment portion.

### BRIEF SUMMARY OF THE INVENTION

According to one aspect of the invention, an operation input unit includes that: a board unit including a switch; a base on which the board unit is disposed; a button portion which is pushed in an operation input; an external force application portion configured to move along a movement axis by the button portion being pushed, and configured to change an open or closed state of the switch by varying an external force which is caused to act on the board unit in accordance with the movement; and a board deflection portion provided in a region in the board unit, where the switch is disposed, the board deflection portion having flexibility, being configured to form a space between the board deflection portion and the base in a state in which the button portion is not pushed, and configured to deflect toward a movement direction of the external force application portion by the external force application portion moving by the pushing of the button portion.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view which schematically illustrates an energy treatment system according to a first embodiment;

FIG. 2 is a cross-sectional view which schematically illustrates, in cross section perpendicular to a width direction of an energy treatment instrument, an operation input unit according to the first embodiment;

FIG. 3 is a schematic view, as viewed from a direction of an arrow III in FIG. 2;

FIG. 4 is a cross-sectional view taken along line IV-IV in FIG. 2;

FIG. 5 is a cross-sectional view which schematically illustrates, in cross section perpendicular to a width direction of a board unit, an operation input unit according to a second embodiment;

FIG. 6 is a cross-sectional view taken along line VT-VI in FIG. 5;

FIG. 7 is a perspective view which schematically illustrates a held unit according to a first modification;

FIG. 8 is a cross-sectional view which schematically illustrates, in cross section perpendicular to the width direction of the board unit, a state in which a moving operation bar is located at a first movement position in an operation input unit according to the first modification;

FIG. 9 is a cross-sectional view which schematically illustrates, in cross section perpendicular to the width direction of the board unit, a state in which the moving operation bar is located at a second movement position in the operation input unit according to the first modification;

FIG. 10 is a cross-sectional view which schematically illustrates, in cross section perpendicular to the width direction of the board unit, an operation input unit according to a first reference example; and

FIG. 11 is a cross-sectional view taken along line XI-XI in FIG. 10.

### DETAILED DESCRIPTION OF THE INVENTION

### First Embodiment

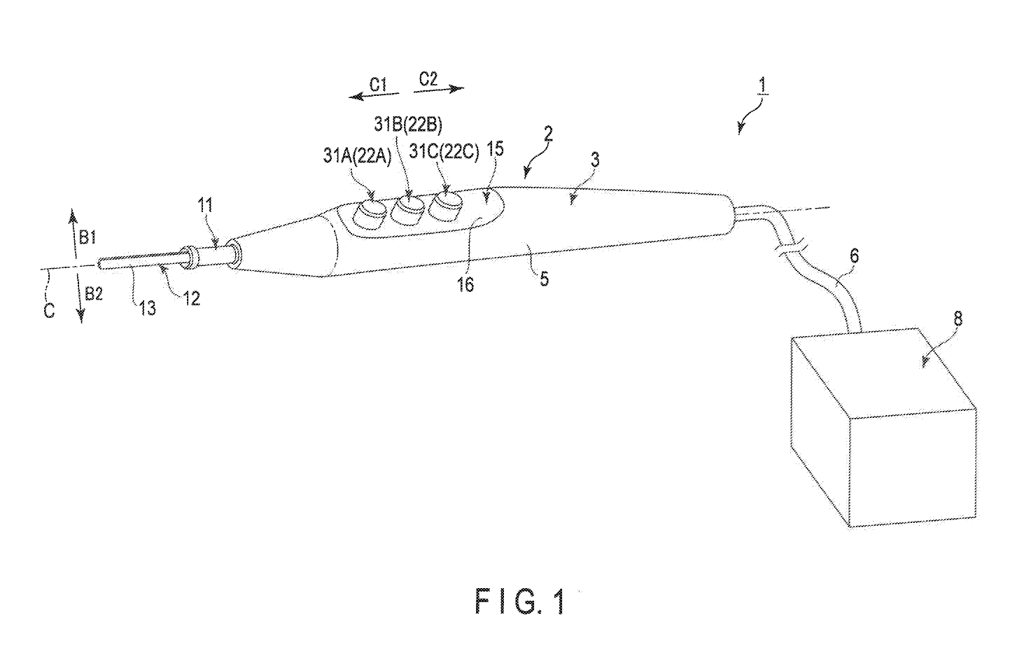

A first embodiment of the present invention will be described with reference to FIG. 1 to FIG. 4.

FIG. 1 is a view illustrating an energy treatment system (energy treatment apparatus) 1. As illustrated in FIG. 1, the energy treatment system 1 includes an energy treatment instrument (high-frequency treatment instrument) 2. The energy treatment instrument 2 has a longitudinal axis C. When a direction parallel to the longitudinal axis C is defined as a longitudinal axial direction, one side in the longitudinal axial direction is a distal side (arrow C1 side in FIG. 1), and a side opposite to the distal side is a proximal side (arrow C2 side in FIG. 1). The energy treatment instrument 2 includes a held unit 3 which extends along the longitudinal axis C. The held unit 3 includes a held casing 5 which forms an armor of the held unit 3. One end of a cable 6 is connected to the held unit 3. The other end of the cable 6 is detachably connected to an energy source unit 8. The energy source unit 8 is, for example, an energy control device. The energy source unit 8 includes an electric power source, a conversion circuit which converts electric power from the electric power source to high-frequency electric power (high-frequency electric energy), and a conversion circuit which converts electric power from the electric power source to vibration generating electric power (vibration generating electric energy). In addition, the energy source unit 8 is provided with a controller which is composed of a processor including a CPU (Central Processing Unit) or an ASIC (application specific integrated circuit) and a storage such as a memory.

In addition, a sheath 11 and a blade 12 are detachably coupled to the held unit 3 from the distal side. The sheath 11 and blade 12 extend along the longitudinal axis C, and are inserted into an inside of the held casing 5 from the distal side. The blade 12 is inserted through the sheath 11, and a distal portion of the blade 12 is provided with a treatment portion (end effector) 13 which projects from a distal end of the sheath 11 toward the distal side. In the inside of the held casing 5, a vibration generating unit (not shown) including an ultrasonic transducer is coupled to the proximal side of the blade 12. In the ultrasonic transducer, vibration generating electric power is supplied from the energy source unit 8, and thereby ultrasonic vibration is generated. The ultrasonic vibration caused by the ultrasonic transducer is transmitted toward the distal side through the blade 12.

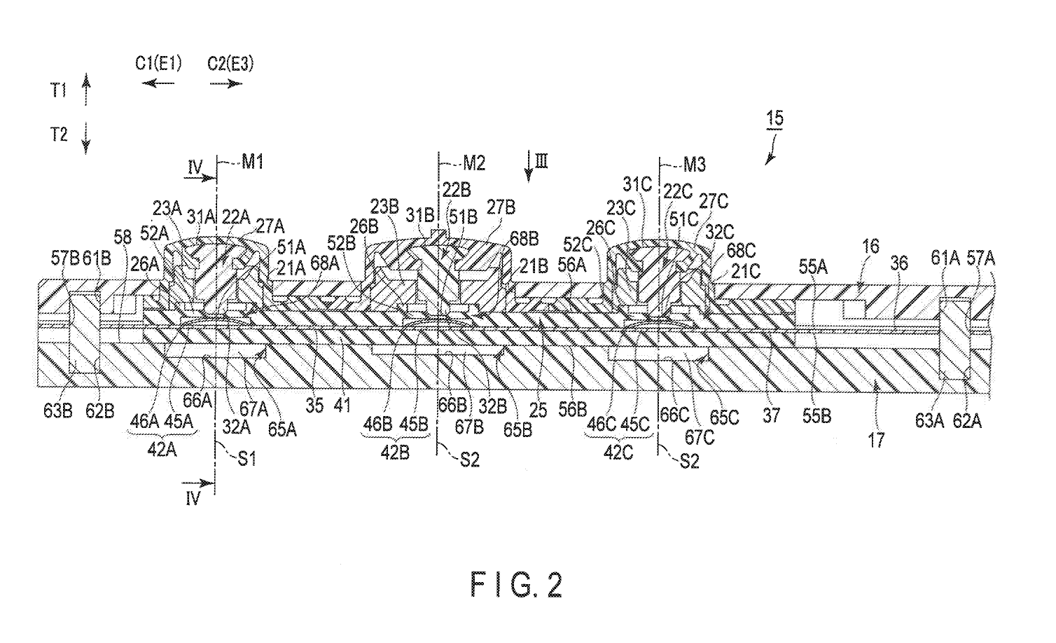

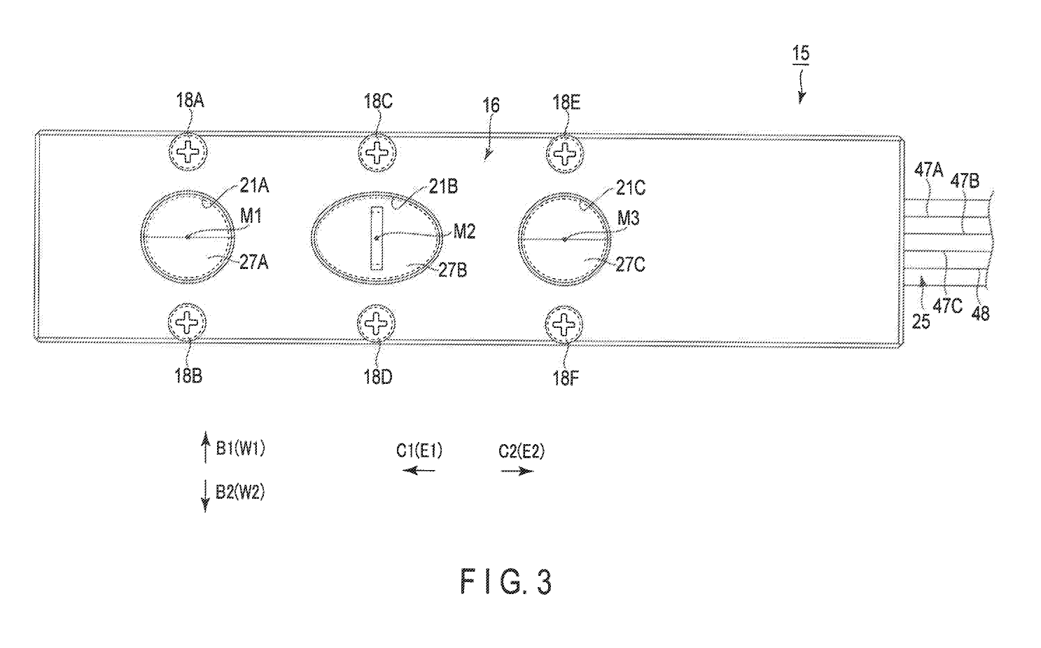

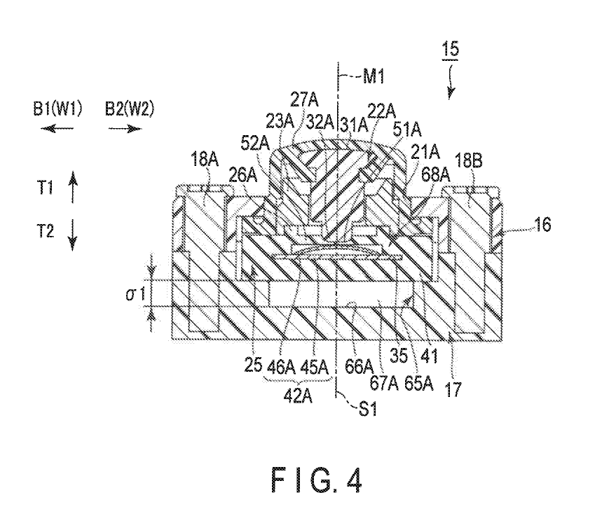

In the held unit 3, there is provided an operation input unit 15 to which an energy operation is input for supplying high-frequency electric power (and ultrasonic vibration) to the treatment portion 13 (blade 12) as energy for use in a treatment. Using the supplied high-frequency electric power, the treatment portion 13 treats a treated target such as a biological tissue. FIG. 2 to FIG. 4 are views illustrating a configuration of the operation input unit 15. Here, a certain direction crossing (perpendicular to) the longitudinal axial direction (directions of an arrow B1 and an arrow B2 in FIG. 1) is defined as a width direction of the energy treatment instrument 2. FIG. 2 illustrates a cross section perpendicular to the width direction of the energy treatment instrument 2. In addition, FIG. 3 is a view, as viewed from a direction of an arrow III in FIG. 2, and FIG. 4 is a cross-sectional view taken along line IV-IV in FIG. 2.

As illustrated in FIG. 2 to FIG. 4, the operation input unit 15 includes a unit armor portion 16 which forms a part of the held casing 5. In addition, the operation input unit 15 includes a base 17 which is disposed in the inside of the held casing 5. The base 17 is fixed to the unit armor portion 16 via fixing pins 18A to 18F (six fixing pins in this embodiment).

In addition, through-holes 21A to 21C (three through-holes in this embodiment), which penetrate from the inside of the held casing 5 to the outside, are formed in the unit armor portion 16. A pusher (corresponding one of 22A to 22C) and a support member (corresponding one of 23A to 23C) are disposed in each of the through-holes 21A to 21C. Each of the pushers (shaft members) 22A to 22C is inserted into the support member (corresponding one of 23A to 23C), and each of the support members 23A to 23C is formed in such a cylindrical shape as to surround the pusher (corresponding one of 22A to 22C). Each of the pushers (moving pushers) 22A to 22C has a movement axis (corresponding one of M1 to M3), and is movable along the movement axis (corresponding one of M1 to M3) relative to the unit armor portion 16, base 17 and support member (corresponding one of 23A to 23C). In the present embodiment, the movement axes M1 to M3 cross (are perpendicular to) the longitudinal axial direction, and cross (are perpendicular to) the width direction of the energy treatment instrument 2.

The operation input unit 15 includes a board unit 25 which is disposed between the unit armor portion 16 and base 17. The board unit 25 extends along an extending direction (directions of arrows E1 and E2 in FIG. 2 and FIG. 3). In this embodiment, the extending direction of the board unit 25 substantially agrees with the longitudinal axial direction (the distal side and proximal side) of the energy treatment instrument 2. In addition, in the board unit 25, a certain direction perpendicular to (crossing) the extending direction is defined as a width direction of the board unit 25 (directions of an arrow W1 and an arrow W2 in FIG. 3 and FIG. 4). Besides, in the board unit 25, a direction perpendicular to (crossing) the extending direction and perpendicular to (crossing) the width direction is defined as a thickness direction of the board unit 25 (directions of an arrow T1 and an arrow T2 in FIG. 2 and FIG. 4). In the present embodiment, the width direction of the board unit 25 substantially agrees with the width direction of the energy treatment instrument 2.

The board unit 25 is disposed on the base 17. The board unit 25 is provided with engaging projection portions 26A to 26C (three engaging projection portions in this embodiment) which project toward the unit armor portion 16. Each of the engaging projection portions 26A to 26C is formed in such a cylindrical shape as to surround the movement axis (corresponding one of M1 to M3) of the pusher (corresponding one of 22A to 22C). Each of the support members 23A to 23C is fixed to the board unit 25 by being engaged with the engaging projection portion (corresponding one of 26A to 26C).

The operation input unit 15 includes cover members 27A to 27C. In each of the through-holes 21A to 21C, the pusher (corresponding one of 22A to 22C) and the support member (corresponding one of 23A to 23C) are covered with the cover member (corresponding one of 27A to 27C). Thus, the pushers 22A to 22C and support members 23A to 23C are not exposed to the outside of the held casing 5. In addition, a part of each of the cover members 27A to 27C, which is located in a part other than the through-hole (corresponding one of 21A to 21C), is clamped between the unit armor portion 16 and board unit 25.

Each of the pushers (shaft members) 22A to 22C includes a button portion (corresponding one of 31A to 31C) which is pushed by a surgeon or the like via the cover member (corresponding one of 27A to 27C) in the input (operation input) of an energy operation. By the button portion (corresponding one of 31A to 31C) being pushed, each of the pushers 22A to 22C moves along the movement axis (corresponding one of M1 to M3). The movement axes M1 to M3 are substantially parallel to the thickness direction of the board unit 25. In addition, each of the pushers 22A to 22C includes an external force application portion (corresponding one of 32A to 32C) which is provided to be capable of pushing the board unit 25 in accordance with a pushing operation of the button portion (corresponding one of 31A to 31C). In each of the pushers 22A to 22C, the external force application portion (corresponding one of 32A to 32C) pushes the board unit 25, thereby exerting an external force on the board unit 25. Besides, in each of the pushers 22A to 22C, the external force application portion (corresponding one of 32A to 32C) moves along the movement axis (corresponding one of M1 to M3) by the button portion (corresponding one of 31A to 31C) being pushed. Furthermore, in each of the pushers 22A to 22C, the pushing state of the board unit 25 by the external force application portion (corresponding one of 32A to 32C) varies by the movement of the external force application portion (corresponding one of 32A to 32C). The external force, which is exerted on the board unit 25 from each of the pushers 22A to 22C, varies in accordance with the pushing state of the board unit 25 by the external force application portion (corresponding one of 32A to 32C).

As illustrated in FIG. 2 to FIG. 4, the board unit 25 includes a switch board 35. The switch board 35 is, for example, a flexible printed board (FPC: flexible printed circuits). The switch board 35 includes an exposed portion 36 which is exposed to the outside of the board unit 25, and a non-exposed portion 37 which is not exposed to the outside of the board unit 25. In addition, the board unit 25 includes a cover portion 41 which covers the non-exposed portion of the switch board 35. The cover portion 41 covers the non-exposed portion 37 from both sides in the thickness direction of the board unit 25. The cover portion 41 is formed of, for example, silicone rubber. In the present embodiment, the entirety of the cover portion 41 is formed of an elastic material.

Besides, in the board unit 25, liquid-tightness is kept between the non-exposed portion 37 of switch board 35 and the cover portion 41. Thus, liquid is prevented from flowing from the outside of the board unit 25 to the non-exposed portion 37 which is located inside the cover portion 41.

The switch board 35 of the board unit 25 includes a board surface (first board surface) 55A which faces the unit armor portion 16 side in the thickness direction of the board unit 25, and a board surface (second board surface) 55B which faces the base 17 side in the thickness direction of the board unit 25. Switches 42A to 42C (three switches in this embodiment) and electrical path portions 47A to 47C, 48 are disposed on the board surface 55A. In addition, the board surface 55B is formed substantially planar. Besides, the cover portion 41 includes a cover portion outer surface (first cover portion outer surface) 56A which faces the unit armor portion 16 side in the thickness direction of the board unit 25, and a cover portion outer surface (second cover portion outer surface) 56B which faces the base 17 side in the thickness direction of the board unit 25. The cover portion outer surface 56B is formed substantially planar.

The switches 42A to 42C are located on the non-exposed portion 37 of the switch board 35. Each of the switches 42A to 42C has a center axis (corresponding one of S1 to S3) along the thickness direction of the board unit 25. The center axis (corresponding one of S1 to S3) of each of the switches 42A to 42C is substantially coaxial with the movement axis (corresponding one of M1 to M3) of the pusher (corresponding one of 22A to 22C). Thus, each of the engaging projection portions 26A to 26C is formed in such a cylindrical shape as to surround the center axis (corresponding one of S1 to S3) of the switch (corresponding one of 42A to 42C). In the meantime, the engaging projection portions 26A to 26C are provided on the cover portion outer surface 56A of the cover portion 41.

Each of the switches 42A to 42C includes a fixed contact portion (corresponding one of 45A to 45C) which is fixed to the switch board 35, and a movable contact portion (corresponding one of 46A to 46C) which is provided to be capable of moving (movable) in the thickness direction of the board unit 25 relative to the fixed contact portion (corresponding one of 45A to 45C). Each of the fixed contact portions (fixed contact point portions) 45A to 45C is located on a side near the base 17 in the thickness direction of the board unit 25 with respect to the movable contact portion (corresponding one of 46A to 46C). The movable contact portions (movable contact point portions) 46A to 46C are formed of a material having elasticity and electrical conductivity, and the cover portion 41 abuts on the movable contact portions 46A to 46C from the unit armor portion 16 side in the thickness direction of the board unit 25.

Besides, on the board surface 55A of the switch board 35, the electrical path portions 47A to 47C, 48 extend along the extending direction of the board unit 25 (in this embodiment, from the proximal side to distal side of the energy treatment instrument 2). Each of the electrical path portions 47A to 47C, 48 is electrically connected to the controller (not shown) of the energy source unit 8 via a corresponding electrical wiring (not shown) which extends through the inside of the cable 6. The electrical path portion 47A is electrically connected to the movable contact portion 46A, and the electrical path portion 47B is electrically connected to the movable contact portion 46B. In addition, the electrical path portion 47C is electrically connected to the movable contact portion 46C. Furthermore, the electrical path portion 48 is electrically connected to all of the fixed contact portions 45A to 45C, and is commonly used as a ground line of all of the switches 42A to 42C.

Each of the external force application portions 32A to 32C (pushers 22A to 22C) abuts on the cover portion outer surface 56A which faces the unit armor portion 16 side in the cover portion 41. Specifically, the cover portion outer surface (first cover portion outer surface) 56A is provided with abutment surface portions (pusher abutment portions) 51A to 51C, and the external force application portion (corresponding one of 32A to 32C) of the pusher (corresponding one of 22A to 22C) abuts on each of the abutment surface portions 51A to 51C. Here, in each of the pushers 22A to 22C, a state in which the button portion (corresponding one of 31A to 31C) is not pushed (a state in which no energy operation is input) is defined as a neutral state (non-pushed state). In each of the pushers 22A to 22C, even in the neutral state (neutral position), the external force application portion (corresponding one of 32A to 32C) abuts on the corresponding abutment surface portion (corresponding one of 51A to 51C). In addition, in each of the pushers 22A to 22C, in the neutral state, a balanced state occurs in which the pushing force (external force) from the external force application portion (corresponding one of 32A to 32C) to the cover portion 41 and the reactive force (elastic force) from the cover portion 41 are balanced. Thus, in the neutral state, each of the pushers 22A to 22C does not move along the movement axis (corresponding one of M1 to M3).

In each of the pushers 22A to 22C, the button portion (corresponding one of 31A to 31C) is pushed (i.e. the energy operation is input), and the external force application portion (corresponding one of 32A to 32C) moves from the neutral state (non-pushed state), and thereby the external force acting on the cover portion 41 varies. The cover portion 41 is provided with elastic deformation portions 52A to 52C. Each of the elastic deformation portions 52A to 52C elastically deforms in accordance with a variation of the external force (pushing force) from the pusher (corresponding one of 22A to 22C). Here, the shape of each of the elastic deformation portions 52A to 52C at a time of the neutral state (non-pushed state) of the corresponding pusher (corresponding one of 22A to 22C) is defined as a neutral shape. Each of the elastic deformation portions 52A to 52C elastically deforms from the neutral shape (toward the arrow T2 side) by the external force (pushing force) from the pusher (corresponding one of 22A to 22C) being increased (varied) by the input of the energy operation.

In addition, in the present embodiment, each of the elastic deformation portions 52A to 52C abuts on the movable contact portion (corresponding one of 46A to 46C) of the corresponding switch (corresponding one of 42A to 42C) from the unit armor portion 16 side. In each of the switches 42A to 42C, when the elastic deformation portion (corresponding one of 52A to 52C) is in the neutral position (i.e. when the pusher (corresponding one of 22A to 22C) is in the neutral state), the movable contact portion (corresponding one of 46A to 46C) is not in contact with the fixed contact portion (corresponding one of 45A to 45C). Each of the elastic deformation portions 52A to 52C elastically deforms from the neutral shape (toward the arrow T2 side), thereby exerting pushing force on the corresponding switch (corresponding one of 42A to 42C). In addition, in each of the switches 42A to 42C, by the pushing force acting from the elastic deformation portion (corresponding one of 52A to 52C) onto the movable contact portion (corresponding one of 46A to 46C), the movable contact portion (corresponding one of 46A to 46C) elastically deforms (toward the arrow T2 side), and the movable contact portion (corresponding one of 46A to 46C) comes in contact with the fixed contact portion (corresponding one of 45A to 45C).

The controller (not shown) of the energy source unit 8 detects the open or closed state of each of the switches 42A to 42C, thereby detecting the presence or absence of the input of the energy operation in the corresponding button portion (corresponding one of 31A to 31C). If the energy operation is input in the button portion 31A and the switch 42A enters the closed state (i.e. if the fixed contact portion 45A and movable contact portion 46A come in contact), the electrical path portion 47A and electrical path portion 48 are electrically connected and electrical conduction is established in the switch 42A. At this time, the energy source unit 8 detects the flow of electric current (detection current) through the electrical path portion 47A and electrical path portion 48, thereby detecting the input of the energy operation in the button portion 31A. In addition, if the energy operation is input in the button portion 31B and the switch 42B enters the closed state, the electrical path portion 47B and electrical path portion 48 are electrically connected in the switch 42B. At this time, the energy source unit 8 detects the flow of electric current (detection current) through the electrical path portion 47B and electrical path portion 48, thereby detecting the input of the energy operation in the button portion 31B. Furthermore, if the energy operation is input in the button portion 31C and the switch 42C enters the closed state, the electrical path portion 47C and electrical path portion 48 are electrically connected in the switch 42C. At this time, the energy source unit 8 detects the flow of electric current (detection current) through the electrical path portion 47C and electrical path portion 48, thereby detecting the input of the energy operation in the button portion 31C. Accordingly, when the switch (corresponding one of 42A to 42C) is in the closed state, electric current, which is supplied to the switch (corresponding one of 42A to 42C), passes through each of the electrical path portions 47A to 47C. Current passes through the electrical path portion 48, when any one of the switches 42A to 42C is in the closed state.

If the input of the energy operation in the button portion 31A of the pusher 22A is detected, a high-frequency electric power is output from the energy source unit 8, and the high-frequency electric power is supplied to the treatment portion 13 (blade 12). In this state, the treatment portion 13 is brought into contact with a treated target such as a biological tissue, and thereby a high-frequency current flows between the treatment portion 13 and a counter-electrode plate (not shown) through the treated target. When the energy operation was input in the button portion 31A, a high-frequency current of a continuous waveform flows to the treated target, and the treated target is cut and opened. Also when the input of the energy operation in the button portion 31C of the pusher 22C was detected, a high-frequency electric power is supplied to the treatment portion 13. However, when the energy operation was input in the button portion 31C, a high-frequency current of not a continuous waveform but a burst waveform flows to the treated target, and the treated target is coagulated. In addition, when the input of the energy operation in the button portion 31B of the pusher 22B was detected, a high-frequency electric power is supplied to the treatment portion 13, and a vibration generating electric power is supplied to an ultrasonic transducer (not shown) and ultrasonic vibration is transmitted to the treatment portion 13. In the treatment portion 13, the treated target is cut and opened by using the ultrasonic vibration, and the high-frequency current of the burst waveform is passed through the treated target and the treated target is coagulated.

If the button portion (corresponding one of 31A to 31C) is no longer pushed by the surgeon (i.e. if the input of the energy operation is released), each of the pushers 22A to 22C returns to the position of the neutral state (balanced state). By the corresponding pusher (corresponding one of 22A to 22C) moving to the neutral state (non-pushed state), each of the elastic deformation portions 52A to 52C restores (elastically restores) to the neutral shape. By the corresponding elastic deformation portion (corresponding one of 52A to 52C) restoring to the neutral shape, the pushing force stops acting on each of the switches 42A to 42C from the elastic deformation portion (corresponding one of 52A to 52C), and the movable contact portion (corresponding one of 46A to 46C) is separated from (comes out of contact with) the fixed contact portion (corresponding one of 45A to 45C). Specifically, by the corresponding elastic deformation portion (corresponding one of 52A to 52C) restoring to the neutral shape, each of the switches 42A to 42C enters the open state. When all of the switches 42A to 42C are in the open state, the supply of high-frequency electric power to the treatment portion 13 from the energy source unit 8 is stopped, and the supply of vibration generating electric power to the ultrasonic transducer is also stopped.

In addition, a first hole 57A and a second hole 57B, which penetrate the switch board 35 in the thickness direction of the board unit 25, are formed in the board unit 25. The first hole 57A and second hole 57B penetrate the switch board 35 from the board surface 55A to the board surface 55B in the exposed portion 36 of the switch board 35. In addition, the second hole 57B is located at a position apart from the first hole 57A in the extending direction of the board unit 25.

In the inner surface of the unit armor portion 16, an engaging groove 61A is provided at a position opposed to the first hole 57A, and an engaging groove 61B is provided at a position opposed to the second hole 57B. In addition, the base 17 includes an installation surface (abutment reception surface) 58 on which the board unit 25 is disposed. In the installation surface 58 of the base 17, an engaging groove 62A is provided at a position opposed to the first hole 57A, and an engaging groove 62B is provided at a position opposed to the second hole 57B. A fixing pin (first fixing pin) 63A, which extends in the thickness direction of the board unit 25, is inserted through the first hole 57A. In addition, one end of the fixing pin 63A is engaged in the engaging groove 61A of the unit armor portion 16, and the other end thereof is engaged in the engaging groove 62A of the base 17. Besides, a fixing pin (second fixing pin) 63B, which extends in the thickness direction of the board unit 25, is inserted through the second hole 57B. In addition, one end of the fixing pin 63B is engaged in the engaging groove 61B of the unit armor portion 16, and the other end thereof is engaged in the engaging groove 62B of the base 17. Accordingly, the board unit 25 is attached to the unit armor portion 16 and base 17 via the fixing pins 63A and 63B.

In the extending direction and width direction, the board unit 25 is positionally set relative to the unit armor portion 16 and base 17. In each of the switches 42A to 42C, the center axis (corresponding one of S1 to S3) is substantially coaxial with the movement axis (corresponding one of M1 to M3) of the corresponding pusher (corresponding one of 22A to 22C). In addition, the cover portion outer surface 56B of the cover portion 41, which is substantially planar, abuts on the installation surface 58 of the base 17. By the cover portion outer surface 56B abutting on the installation surface (abutment reception surface) 58 of the base 17, the board unit 25 is positionally set relative to the unit armor portion 16 and base 17 in the thickness direction. The cover portion outer surface 56B abuts on the installation surface 58, also in the state in which all the elastic deformation portions 52A to 52C are in the neutral shape (i.e. also in the state in which none of the button portions 32A to 32C is pushed).

Furthermore, the base 17 is provided with recess portions 65A to 65C (three recess portions in this embodiment) which are recessed from the installation surface (abutment reception surface) 58. Each of the recess portions 65A to 65C is provided in a position where the center axis (corresponding one of S1 to S3) of the corresponding switch (corresponding one of 42A to 42C) and the movement axis (corresponding one of M1 to M3) of the corresponding pusher (corresponding one of 22A to 22C) pass. Each of the recess portions 65A to 65C is recessed from the installation surface 58 in the direction of movement (i.e. the arrow T2 side) of the external force application portion (corresponding one of 32A to 32C) in the state in which the button portion (corresponding one of 31A to 31C) was pushed in the corresponding pusher (corresponding one of 22A to 22C). Each of the recess portions 65A to 65C includes a recess portion bottom surface (corresponding one of 66A to 66C) which is opposed to the cover portion outer surface 56B of the cover portion 41. The recess portion bottom surface (corresponding one of 66A to 66C) of each of the recess portions 65A to 65C has a space (corresponding one of 67A to 67C) between the recess portion bottom surface (corresponding one of 66A to 66C) and the board unit 25 (cover portion 41), in the neutral state (non-pushed state) in which the button portion (corresponding one of 31A to 31C) of the corresponding pusher (corresponding one of 22A to 22C) is not pushed (i.e. when the corresponding elastic deformation portion (corresponding one of 52A to 52C) is in the neutral shape).

The board unit 25 is provided with board deflection portions 68A to 68C (three board deflection portions in this embodiment). Each of the board deflection portions 68A to 68C is provided in a region of the board unit 25, where the corresponding switch (corresponding one of 42A to 42C) and the corresponding elastic deformation portion (corresponding one of 52A to 52C) are located. Specifically, each of the board deflection portions 68A to 68C includes the switch (corresponding one of 42A to 42C) and the elastic deformation portion (corresponding one of 52A to 52C). Thus, each of the board deflection portions 68A to 68C is provided in a position where the center axis (corresponding one of S1 to S3) of the corresponding switch (corresponding one of 42A to 42C) and the movement axis (corresponding one of M1 to M3) of the corresponding pusher (corresponding one of 22A to 22C) pass. In addition, each of the board deflection portions 68A to 68C is formed of a part of the switch board 35 and a part of the cover portion 41. In the present embodiment, the switch board 35 is a flexible printed board, and the cover portion 41 is formed of an elastic material. Thus, the board deflection portions 68A to 68C have flexibility.

Each of the board deflection portions 68A to 68C is provided on the unit armor portion 16 side with respect to the recess portion bottom surface (corresponding one of 66A to 66C) of the corresponding recess portion (corresponding one of 65A to 65C) of the base 17. Thus, each of the board deflection portions 68A to 68C is opposed to the recess portion bottom surface (corresponding one of 66A to 66C) of the corresponding recess portion (corresponding one of 65A to 65C). In addition, in the neutral state (non-pushed state) in which the button portion (corresponding one of 31A to 31C) of the corresponding pusher (corresponding one of 22A to 22C) is not pushed, the space (corresponding one of 67A to 67C) is formed between each of the board deflection portions 68A to 68C and the recess portion bottom surface (corresponding one of 66A to 66C) of the corresponding recess portion (corresponding one of 65A to 65C).

As described above, in the present embodiment, the space (corresponding one of 67A to 67C) is formed in association with each of the board deflection portions 68A to 68C. Thus, each of the board deflection portions 68A to 68C deflects toward the base 17 side, by the external force application portion (corresponding one of 32A to 32C) moving toward the base 17 side from the neutral state by the pushing of the button portion (corresponding one of 31A to 31C) in the corresponding pusher (corresponding one of 22A to 22C). Specifically, by the button portion (corresponding one of 31A to 31C) being pushed in the corresponding pusher (corresponding one of 22A to 22C), each of the board deflection portions 68A to 68C deflects toward the movement direction (the arrow T2 side) of the external force application portion (corresponding one of 32A to 32C) from the neutral state (non-pushed state).

By deflecting toward the base 17 side, each of the board deflection portions 68A to 68C abuts on the recess portion bottom surface (corresponding one of 66A to 66C) of the corresponding recess portion (corresponding one of 65A to 65C). By abutting on the corresponding recess portion bottom surface (corresponding one of 66A to 66C), each of the board deflection portions 68A to 68C is prevented from further deflecting toward the base 17 side. Accordingly, in each of the recess portions 65A to 65C, the recess portion bottom surface (corresponding one of 66A to 66C) functions as a deflection amount restriction portion configured to restrict a deflection amount (corresponding one of δ1 to δ3) of the corresponding board deflection portion (corresponding one of 68A to 68C) in the state in which the corresponding board deflection portion (corresponding one of 68A to 68C) has deflected toward the base 17 side. Specifically, in the state in which each of the board deflection portions 68A to 68C has deflected toward the movement direction of the corresponding external force application portion (corresponding one of 32A to 32C) from the non-pushed state, each of the board deflection portions 68A to 68C abuts on the recess portion bottom surface (corresponding one of 66A to 66C) of the corresponding recess portion (corresponding one of 65A to 65C), and thereby the deflection amount (corresponding one of δ1 to δ3) is restricted. In each of the board deflection portions 68A to 68C, the deflection amount (corresponding one of δ1 to δ3) is adjusted by adjusting a recess dimension (corresponding one of σ1 to σ3) from the installation surface (abutment reception surface) 58 to the recess portion bottom surface (corresponding one of 66A to 66C) in the corresponding recess portion (corresponding one of 65A to 65C).

By the corresponding pusher (corresponding one of 22A to 22C) moving to the neutral state (non-pushed state), each of the board deflection portions 68A to 68C transitions (elastically restores) to the state in which the board deflection portion does not deflect toward the base 17 side. Thereby, each of the board deflection portions 68A to 68C is spaced apart from the recess portion bottom surface (corresponding one of 66A to 66C) of the corresponding recess portion (corresponding one of 65A to 65C), and the space (corresponding one of 67A to 67C) is formed between each of the board deflection portions 68A to 68C and the corresponding recess portion bottom surface (corresponding one of 66A to 66C).

Next, the functions and advantageous effects of the operation input unit 15 and energy treatment instrument 2 of the present embodiment will be described. When a treatment is performed by using the energy treatment instrument 2, the sheath 11 and blade 12 are inserted into the body. Then, the treatment portion 13 of the blade 12 is put in contact with the treated target. In this state, in any one of the pushers 22A to 22C, the button portion (one of 31A to 31C) is pushed, and an energy operation is input. Thereby, the switch (corresponding one of 42A to 42C), which corresponds to the pushed button portion (one of 31A to 31C), enters the closed state, and the input of the energy operation is detected by the energy source unit 8. By the input of the energy operation in any one of the button portions 31A to 31C being detected, high-frequency electric power is supplied from the energy source unit 8 to the treatment portion 13, and the treatment portion 13 treats the treated target by using the supplied high-frequency electric power. In the meantime, when an energy operation was input by the button portion 31B, high-frequency electric power is supplied to the treatment portion 13, and ultrasonic vibration is transmitted to the treatment portion 13.

In the pusher (corresponding one of 22A to 22C) in which the button portion (corresponding of 31A to 31C) was pushed, the external force application portion (corresponding one of 32A to 32C) moves from the neutral state, and the external force acting on the cover portion 41 varies. Thereby, the elastic deformation portion (corresponding one of 52A to 52C), which corresponds to the pusher (corresponding one of 22A to 22C) that has moved from the neutral state, elastically deforms from the neutral shape. Then, a pushing force acts on the corresponding switch (corresponding one of 42A to 42C) from the elastic deformation portion (corresponding one of 52A to 52C) which has elastically deformed from the neutral shape. The switch (corresponding one of 42A to 42C), on which the pushing force acts from the corresponding elastic deformation portion (corresponding one of 52A to 52C), enters the closed state, by the movable contact portion (corresponding one of 46A to 46C) coming in contact with the fixed contact portion (corresponding one of 45A to 45C).

In addition, in the present embodiment, in the pusher (corresponding one of 22A to 22C) in which the button portion (corresponding of 31A to 31C) was pushed, the external force application portion (corresponding one of 32A to 32C) moves toward the base 17 side from the neutral state. Thereby, the corresponding board deflection portion (corresponding one of 68A to 68C) deflects toward the movement direction (the base 17 side) of the external force application portion (corresponding one of 32A to 32C). By deflecting toward the base 17 side, each of the board deflection portions 68A to 68C deflects until abutting on the recess portion bottom surface (corresponding one of 66A to 66C) of the corresponding recess portion (corresponding one of 65A to 65C). Here, in each of the pushers 22A to 22C, a stroke (corresponding one of P1 to P3) of the external force application portion (corresponding one of 32A to 32C) is defined, the stroke being in a range from the neutral state (non-pushed state), in which the button portion (corresponding one of 31A to 31C) is not pushed, to the closed state of the corresponding switch (corresponding one of 42A to 42C). In addition, in each of the switches 42A to 42C, there is defined a movement amount (corresponding one of Y1 to Y3) of the movable contact portion (corresponding one of 46A to 46C) relative to the fixed contact portion (corresponding one of 45A to 45C), from the non-pushed state of the corresponding pusher (corresponding one of 22A to 22C) to the closed state. In the range from the non-pushed state of the pusher 22A (external force application portion 32A) to the closed state of the switch 42A, equation (1) is established by using the deflection amount δ1 of the board deflection portion 68A, the stroke (movement amount) P1 of the external force application portion 32A, and the movement amount Y1 of the movable contact portion 46A relative to the fixed contact portion 45A.

(Equation 1)

P1=Y1+δ1 (1)

The same relationship as equation (1) is established, also in the range from the non-pushed state of the pusher 22B (external force application portion 32B) to the closed state of the switch 42B, and in the range from the non-pushed state of the pusher 22C (external force application portion 32C) to the closed state of the switch 42C. Accordingly, in the present embodiment, in each of the pushers 22A to 22C, the stroke (corresponding one of P1 to P3) of the external force application portion (corresponding one of 32A to 32C) from the non-pushed state (neutral state) to the closed state of the corresponding switch (corresponding one of 42A to 42C) is adjusted by adjusting the deflection amount (corresponding one of δ1 to δ3) of the corresponding board deflection portion (corresponding one of 68A to 68C). Thus, in each of the pushers 22A to 22C, the stroke (corresponding one of P1 to P3) of the external force application portion (corresponding one of 32A to 32C) is adjusted, regardless of the movement amount (corresponding one of Y1 to Y3) of the movable contact portion (corresponding one of 46A to 46C) relative to the fixed contact portion (corresponding one of 45A to 45C) in the corresponding switch (corresponding one of 42A to 42C) in the range from the non-pushed state (neutral state) to the closed state of the corresponding switch (corresponding one of 42A to 42C). Specifically, regardless of the specifications of the corresponding switch (corresponding one of 42A to 42C), by adjusting the deflection amount (corresponding one of δ1 to δ3) of the corresponding board deflection portion (corresponding one of 68A to 68C), the stroke (corresponding one of P1 to P3) of the external force application portion (corresponding one of 32A to 32C) is adjusted in each of the pushers 22A to 22C.

In addition, in each of the board deflection portions 68A to 68C, the deflection amount (corresponding one of δ1 to δ3) is adjusted by adjusting the recess dimension (corresponding one of σ1 to σ3) from the installation surface (abutment reception surface) 58 to the recess portion bottom surface (corresponding one of 66A to 66C) in the corresponding recess portion (corresponding one of 65A to 65C). Accordingly, in the present embodiment, by adjusting the recess dimension (corresponding one of σ1 to σ3) of the corresponding recess portion (corresponding one of 65A to 65C), the stroke (corresponding one of P1 to P3) of the external force application portion (corresponding one of 32A to 32C) is adjusted in each of the pushers 22A to 22C.

For example, when the dimension of the board unit 25 (switch board 35) is small in the thickness direction, there is a case in which one certain switch 35A is reduced in size. When the small-sized switch 35A is used, the movement amount Y1 of the movable contact portion 46A relative to the fixed contact portion 45A, from the neutral state of the button portion 31A to the closed state of the switch 42A, becomes smaller. However, in this embodiment, even if the movement amount Y1 of the movable contact portion 46A relative to the fixed contact portion 45A is small, it is possible to increase the stroke P1 of the external force application portion 32A of the pusher 22A from the neutral state of the pusher 22A to the closed state of the switch 42A, by adjusting the recess dimension σ1 of the recess portion 65A and thereby adjusting the deflection amount δ1 of the board deflection portion 68A. Similarly, the stroke P2 of the external force application portion 32B of the pusher 22B can be increased even when the switch 42B is reduced in size, and the stroke P3 of the external force application portion 32C of the pusher 22C can be increased even when the switch 42C is reduced in size.

Accordingly, in the present embodiment, by adjusting the deflection amount (corresponding one of δ1 to δ3) of the corresponding board deflection portion (corresponding one of 68A to 68C) (i.e. by adjusting the recess dimension (corresponding one of σ1 to σ3) of the corresponding recess portion (corresponding one of 65A to 65C)), the stroke (corresponding one of P1 to P3) of the external force application portion (corresponding one of 32A to 32C) can be set appropriately for the operator (surgeon) in each of the pushers 22A to 22C. Thereby, regardless of the specifications of the corresponding switch (corresponding one of 42A to 42C), the stroke (corresponding one of P1 to P3) of the external force application portion (corresponding one of 32A to 32C) can be properly set in each of the pushers 22A to 22C, and the operability at a time when an operation input was executed by the operator can be secured.

Furthermore, by deflecting (elastically deforming), each of the board deflection portions 68A to 68C causes a reactive force (elastic force) in such a direction as to return to the non-deflected state. The reactive force from the corresponding board deflection portion (corresponding one of 68A to 68C) is transmitted to the button portion (corresponding one of 31A to 31C) via the external force application portion (corresponding one of 32A to 32C) in each of the pushers 22A to 22C. In each of the pushers 22A to 22C, by the reactive force from the corresponding board deflection portion (corresponding one of 68A to 68C) being transmitted to the button portion (corresponding one of 31A to 31C), the click feeling of the operator, who is pushing the button portion (corresponding one of 31A to 31C), is improved. Thereby, the operability at a time when the operation input was executed by the operator can be improved.

### Second Embodiment

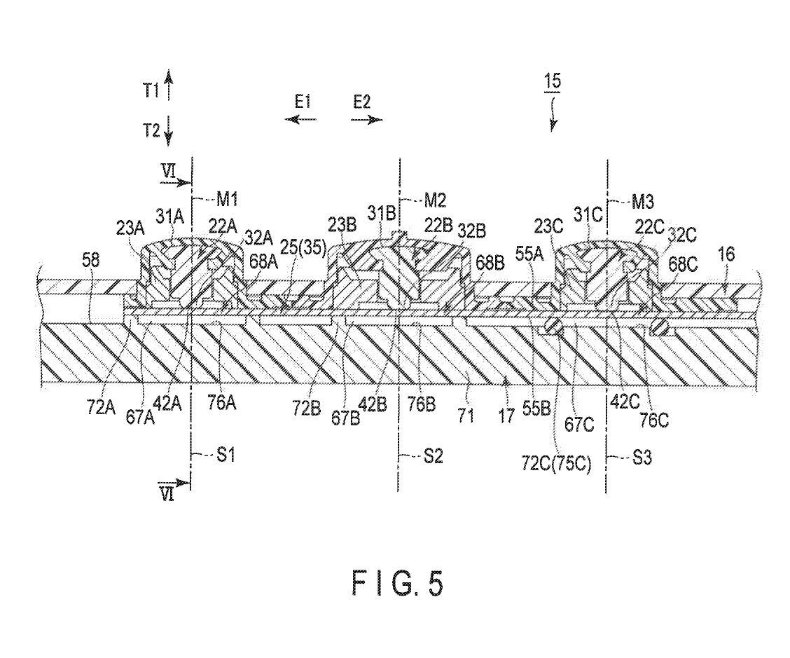

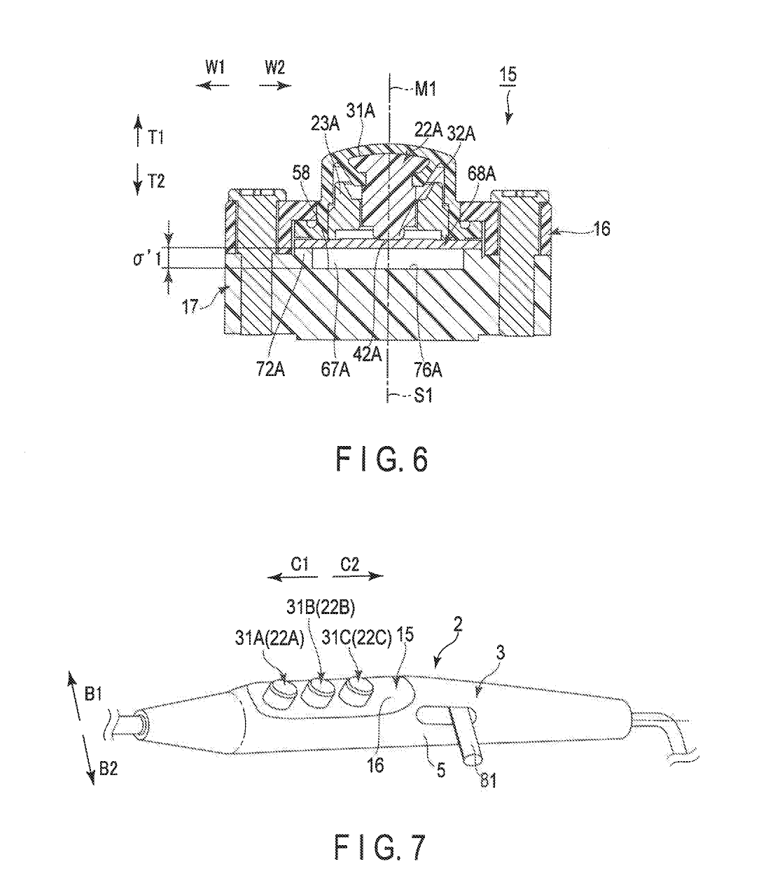

Next, a second embodiment of the present invention will be described with reference to FIG. 5 and FIG. 6. In the second embodiment, the configuration of the first embodiment is modified as described below. Incidentally, the same parts as in the first embodiment are denoted by like reference numerals, and a description thereof is omitted.

FIG. 5 and FIG. 6 illustrate an operation input unit 15. FIG. 5 illustrates a cross section perpendicular to the width direction of the energy treatment instrument 2 (the width direction of the board unit 25). In addition, FIG. 6 is a cross-sectional view taken along line VI-VI in FIG. 5. As illustrated in FIG. 5 and FIG. 6, in the present embodiment, the board unit 25 is formed of only the switch board 35. Thus, the entirety of the switch board 35 is exposed to the outside of the board unit 25. In this embodiment, the base 17 includes a base body 71, and the base body 71 is provided with an installation surface 58 on which the board is disposed. In addition, the base 17 is provided with support portions 72A to 72C (three support portions in this embodiment) which support the board unit 25 in the state in which the support portions 72A to 72C abut on the board surface 55B of the switch board 35. Each of the support portions 72A to 72C is formed in such a cylindrical shape as to surround the center axis (corresponding one of S1 to S3) of the corresponding switch (corresponding one of 42A to 42C). In the present embodiment, the support portions 72A and 72B are provided integral with the base body 71, and the support portion 72C is formed of an elastic portion (elastic member) 75C which is detachably attached to the base body 71. The board unit 25 (switch board 35) is supported by the support portions 72A to 72C. Thereby, in the state in which none of the button portions 31A to 31C is pushed, the board surface 55B of the switch board 35 is spaced apart from the installation surface 58 of the base 17 (i.e. not in contact with the installation surface 58).

In the present embodiment, too, the board unit 25 is provided with board deflection portions 68A to 68C. Each of the board deflection portions 68A to 68C is provided in a region where the corresponding switch (corresponding one of 42A to 42C) is located. Thus, each of the board deflection portions 68A to 68C is provided at a position where the center axis (corresponding one of S1 to S3) of the corresponding switch (corresponding one of 42A to 42C) and the movement axis (corresponding one of M1 to M3) of the corresponding pusher (corresponding one of 22A to 22C) pass. Each of the board deflection portions 68A to 68C is formed of a part of the switch board 35. Since the switch board 35 is a flexible printed board (FPC), the board deflection portions 68A to 68C have flexibility.

In addition, in this embodiment, the installation surface 58 of the base body 71 (base 17) is provided with separate counter-surfaces 76A to 76C, each of which is opposed to the corresponding board deflection portion (corresponding one of 68A to 68C). Specifically, each of the board deflection portions 68A to 68C is provided on the unit armor portion 16 side with respect to the corresponding separate counter-surface (corresponding one of 76A to 76C) of the base 17. In addition, each of the board deflection portions 68A to 68C is opposed to the corresponding separate counter-surface (corresponding one of 76A to 76C). Moreover, in the neutral state (non-pushed state) in which the button portion (corresponding one of 31A to 31C) of the corresponding pusher (corresponding one of 22A to 22C) is not pushed, a space (corresponding one of 67A to 67C) is formed between each of the board deflection portions 68A to 68C and the corresponding separate counter-surface (corresponding one of 76A to 76C).

Accordingly, in this embodiment, too, the space (corresponding one of 67A to 67C) is formed in association with each of the board deflection portions 68A to 68C. Thus, each of the board deflection portions 68A to 68C deflects toward the base 17 side, by the external force application portion (corresponding one of 32A to 32C) moving toward the base 17 side from the neutral state by the pushing of the button portion (corresponding one of 31A to 31C) in the corresponding pusher (corresponding one of 22A to 22C). Specifically, by the button portion (corresponding one of 31A to 31C) being pushed in the corresponding pusher (corresponding one of 22A to 22C), each of the board deflection portions 68A to 68C deflects toward the movement direction (the arrow T2 side) of the external force application portion (corresponding one of 32A to 32C) from the neutral state (non-pushed state).

By deflecting toward the base 17 side, each of the board deflection portions 68A to 68C abuts on the corresponding separate counter-surface (corresponding one of 76A to 76C). By abutting on the corresponding separate counter-surface (corresponding one of 76A to 76C), each of the board deflection portions 68A to 68C is prevented from further deflecting toward the base 17 side. Accordingly, each of the separate counter-surfaces 76A to 76C functions as a deflection amount restriction portion configured to restrict a deflection amount (corresponding one of δ1 to δ3) of the corresponding board deflection portion (corresponding one of 68A to 68C) in the state in which the corresponding board deflection portion (corresponding one of 68A to 68C) has deflected toward the base 17 side. Specifically, in the state in which each of the board deflection portions 68A to 68C has deflected toward the movement direction of the corresponding external force application portion (corresponding one of 32A to 32C) from the non-pushed state, each of the board deflection portions 68A to 68C abuts on the corresponding separate counter-surface (corresponding one of 76A to 76C), and thereby the deflection amount (corresponding one of δ1 to δ3) is restricted. In each of the board deflection portions 68A to 68C, the deflection amount (corresponding one of δ1 to δ3) is adjusted by adjusting a separation dimension (corresponding one of σ′1 to σ′3) to the corresponding separate counter-surface (corresponding one of 76A to 76C) in the non-pushed state of the corresponding pusher (corresponding one of 22A to 22C).

In addition, the elastic portion 75C (support portion 72C) is pushed from the board deflection portion 68C, by the board deflection portion 68C deflecting in the direction of movement of the external force application portion 32C. Thereby, the elastic portion 75C elastically contracts.

By the corresponding pusher (corresponding one of 22A to 22C) moving to the neutral state (non-pushed state), each of the board deflection portions 68A to 68C transitions to the state in which the board deflection portion does not deflect toward the base 17 side. Thereby, each of the board deflection portions 68A to 68C is spaced apart from the corresponding separate counter-surface (corresponding one of 76A to 76C), and the space (corresponding one of 67A to 67C) is formed between each of the board deflection portions 68A to 68C and the corresponding separate counter-surface (corresponding one of 76A to 76C). In addition, by the board deflection portion 68C transitioning to the state in which the board deflection portion 68C does not deflect, the elastic portion 75C is no longer pushed by the board deflection portion 68C. Thereby, the elastic portion 75C elastically restores (elastically restores to the non-contracted state).

Because of the above-described configuration, in the present embodiment, too, the above-described equation (1) is established in the range from the non-pushed state of the pusher 22A (external force application portion 32A) to the closed state of the switch 42A. The same relationship as equation (1) holds true, also in the range from the non-pushed state of the pusher 22B (external force application portion 32B) to the closed state of the switch 42B. In addition, by the board deflection portion 68C deflecting in the direction of movement of the external force application portion 32C, the elastic portion 75C (support portion 72C) elastically contracts. Thus, if a contraction amount ε3 of the elastic portion 75C in the range from the non-pushed state of the pusher 22C (external force application portion 32C) to the closed state of the switch 42C is defined, equation (2) is established.

(Equation 2)

P3=Y3+δ3+ε3 (2)

Accordingly, in the present embodiment, too, in each of the pushers 22A to 22C, the stroke (corresponding one of P1 to P3) of the external force application portion (corresponding one of 32A to 32C) from the non-pushed state (neutral state) to the closed state of the corresponding switch (corresponding one of 42A to 42C) is adjusted by adjusting the deflection amount (corresponding one of δ1 to δ3) of the corresponding board deflection portion (corresponding one of 68A to 68C). Specifically, regardless of the specifications of the corresponding switch (corresponding one of 42A to 42C), by adjusting the deflection amount (corresponding one of δ1 to δ3) of the corresponding board deflection portion (corresponding one of 68A to 68C), the stroke (corresponding one of P1 to P3) of the external force application portion (corresponding one of 32A to 32C) is adjusted in each of the pushers 22A to 22C.

In addition, in each of the board deflection portions 68A to 68C, the deflection amount (corresponding one of δ1 to δ3) is adjusted by adjusting the separation dimension (corresponding one of σ′1 to σ′3) to the corresponding separate counter-surface (corresponding one of 76A to 76C) in the non-pushed state of the corresponding pusher (corresponding one of 22A to 22C). Accordingly, in the present embodiment, by adjusting the separation dimension (corresponding one of σ′1 to σ′3) to the corresponding separate counter-surface (corresponding one of 76A to 76C) from the corresponding board deflection portion (corresponding one of 68A to 68C) in the non-pushed state, the stroke (corresponding one of P1 to P3) of the external force application portion (corresponding one of 32A to 32C) is adjusted in each of the pushers 22A to 22C.

As described above, in the present embodiment, like the first embodiment, by adjusting the deflection amount (corresponding one of δ1 to δ3) of the corresponding board deflection portion (corresponding one of 68A to 68C), the stroke (corresponding one of P1 to P3) of the external force application portion (corresponding one of 32A to 32C) can be set appropriately for the operator (surgeon) in each of the pushers 22A to 22C. Thereby, regardless of the specifications of the corresponding switch (corresponding one of 42A to 42C), the stroke (corresponding one of P1 to P3) of the external force application portion (corresponding one of 32A to 32C) can be properly set in each of the pushers 22A to 22C, and the operability at a time when an operation input was executed by the operator can be secured.

Furthermore, in this embodiment, like the first embodiment, in each of the pushers 22A to 22C, by the reactive force from the corresponding board deflection portion (corresponding one of 68A to 68C) being transmitted to the button portion (corresponding one of 31A to 31C), the click feeling of the operator, who is pushing the button portion (corresponding one of 31A to 31C), is improved. Thereby, the operability at a time when the operation input was executed by the operator can be improved.

Besides, in the present embodiment, by the board deflection portion 68C deflecting, the elastic portion 75C (support portion 72C) elastically contracts. Thus, in the range from the non-pushed state of the pusher 22C (external force application portion 32C) to the closed state of the switch 42C, even if the deflection amount δ3 of the board deflection portion 68C is decreased, the stroke P3 of the external force application portion 32C (pusher 22C) can be increased by increasing the contraction amount ε3 of the elastic portion 75C. By the deflection amount δ3 of the board deflection portion 68C decreasing, it is possible to decrease the load on the board unit 25 (switch board 35) in the state in which the board deflection portion 68C has deflected.

(Modifications)

In the meantime, in a certain modification, in the configuration in which the base 17 is provided with the support portions 72A to 72C as in the second embodiment, each of the support portions 72A and 72B may also be formed of an elastic portion (corresponding one of 75A and 75B). In the present modification, each of the support portions 72A and 72B (elastic portions 75A and 75B) elastically contracts, by the deflection of the corresponding board deflection portion (corresponding one of 68A and 68B) toward the direction of movement of the corresponding external force application portion (corresponding one of 32A and 32B).

In another modification, in the configuration in which the base 17 is provided with the support portions 72A to 72C as in the second embodiment, the board unit 25, which is provided with the switch board 35 and cover portion 41 as in the first embodiment, may be supported by the support portions 72A to 72C. In the present modification, the support portions 72A to 72C abut on the cover portion outer surface 56B of the cover portion 41. In addition, the board unit 25 is, like the first embodiment, provided with board deflection portions 68A to 68C, and each of the board deflection portions 68A to 68C is, like the second embodiment, opposed to the corresponding separate counter-surface (corresponding one of 76A to 76C). In addition, like the second embodiment, in the neutral state (non-pushed state) in which the button portion (corresponding one of 31A to 31C) of the corresponding pusher (corresponding one of 22A to 22C) is not pushed, the space (corresponding one of 67A to 67C) is formed between each of the board deflection portions 68A to 68C and the corresponding separate counter-surface (corresponding one of 76A to 76C).

In still another modification, in the configuration in which the base is provided with the recess portions 65A to 65C as in the first embodiment, the board unit 25, which is not provided with the cover portion 41 as in the second embodiment (i.e. which is formed of only the switch board 35), may be disposed on the installation surface 58 of the base 17. In the present modification, the board surface 55B of the switch board 35 abuts on the installation surface (abutment reception surface) 58. The board unit 25 (switch board 35) is, like the second embodiment, provided with board deflection portions 68A to 68C, and each of the board deflection portions 68A to 68C is, like the first embodiment, opposed to the recess portion bottom surface (corresponding one of 66A to 66C) of the corresponding recess portion (corresponding one of 65A to 65C). In addition, like the first embodiment, in the neutral state (non-pushed state) in which the button portion (corresponding one of 31A to 31C) of the corresponding pusher (corresponding one of 22A to 22C) is not pushed, the space (corresponding one of 67A to 67C) is formed between each of the board deflection portions 68A to 68C and the corresponding recess portion bottom surface (corresponding one of 66A to 66C).

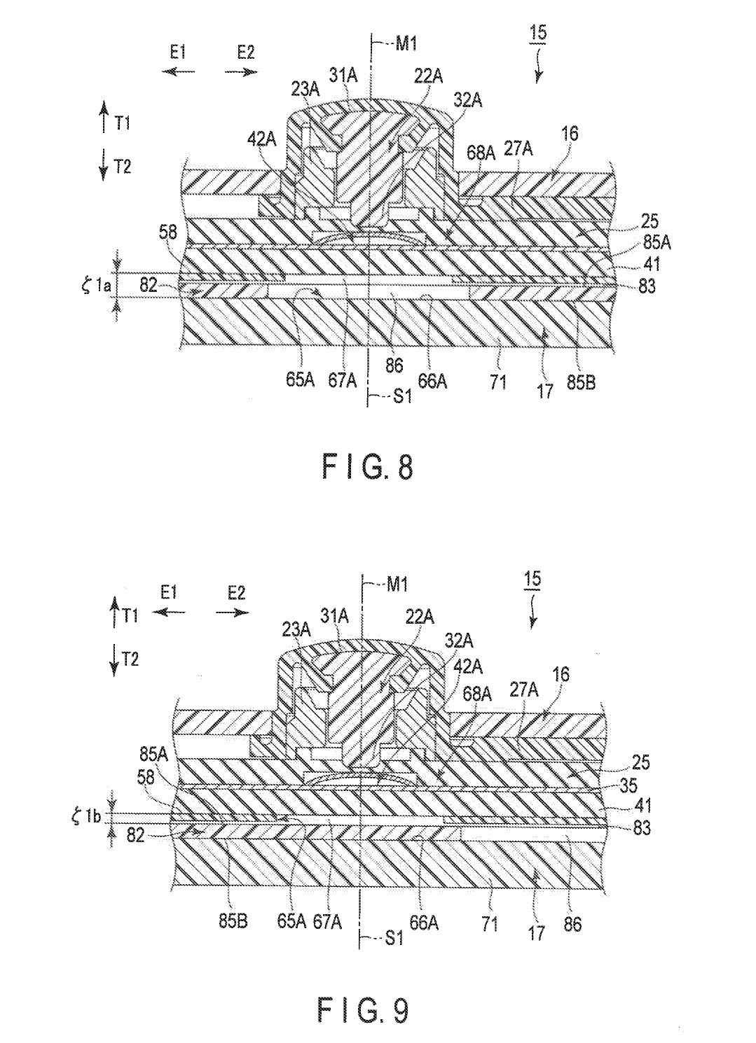

Besides, in a first modification illustrated in FIG. 7 to FIG. 9, the operation input unit 15 is provided with a moving operation bar 81 which is a distance adjusting portion. Here, FIG. 7 is a view illustrating a held unit 3, and FIG. 8 and FIG. 9 illustrate the operation input unit 15 in cross section perpendicular to the width direction of the board unit 25. As illustrated in FIG. 7, the moving operation bar 81 is attached to the held casing 5, and is movable relative to the held casing 5 along the longitudinal axial direction. In addition, the base 17 includes a base body 71, and a moving portion 82 which is provided movable relative to the base body 71 in the extending direction (longitudinal axial direction) of the board unit 25. A moving operation for moving the moving portion 82 relative to the base body 71 is input by the moving operation bar 81.

As illustrated in FIG. 8 and FIG. 9, in the present modification, like the first embodiment, the recess portions 65A to 65C are formed in the base 17 (base body 71). In addition, in the present modification, an inner cavity 83 is formed in the base body 71, and the inner cavity 83 communicates with the space (corresponding one of 67A to 67C) in each of the recess portions 65A to 65C. In the inner cavity 83, the moving portion 82 is movable relative to the base body 71 in the extending direction of the board unit 25. Based on the moving operation by the moving operation bar 81, the moving portion 82 is movable between a first movement position illustrated in FIG. 8 and a second movement position illustrated in FIG. 9.

The moving portion 82 includes a moving portion outer surface (first moving portion outer surface) 85A facing the unit armor portion 16 side, and a moving portion outer surface (second moving portion outer surface) 85B facing the side opposite to the moving portion outer surface 85A. In addition, a through-hole 86, which penetrates from the moving portion outer surface 85A to the moving portion outer surface 85B, is formed in the moving portion 82.

As illustrated in FIG. 8, in the state in which the moving portion 82 is located at the first movement position, the through-hole 86 is located in the recess portion 65A in the base 17. Thus, in the recess portion 65A, the recess portion bottom surface 66A is opposed to the board deflection portion 68A of the board unit 25 through the through-hole 86. In the state in which the board deflection portion 68A does not deflect, the distance between the board deflection portion 68A and base 17 (recess portion bottom surface 66A) in the space 67A is a distance (first distance) ζ1a. As described above, the board deflection portion 68A deflects toward the movement direction of the external force application portion 32A from the neutral state (non-pushed state). At this time, in the state in which the moving portion 82 is at the first movement position, the board deflection portion 68A deflects until abutting on the recess portion bottom surface 66A of the recess portion 65A through the through-hole 86 of the moving portion 82, and deflects by a deflection amount (first deflection amount) δ1a.

If the moving portion 82 is moved to the second movement position along the extending direction of the board unit 25 by the moving operation with the moving operation bar (distance adjusting portion) 81, the through-hole 86 is located at a position apart from the recess portion 65A. Thus, in the recess portion 65A, the moving portion 82 intervenes between the recess portion bottom surface 66A and the board deflection portion 68A of the board unit 25. Accordingly, in the state in which the moving portion 82 is located at the second movement position, the moving portion outer surface 85A of the moving portion 82 is opposed to the board deflection portion 68A. Since the moving portion 82 intervenes, in the state in which the board deflection portion 68A does not deflect, the distance between the board deflection portion 68A and the base 17 (moving portion outer surface 85A) in the space 67A is a distance (second distance) ζ1b which is less than the distance ζ1a. In the state in which the moving portion 82 is at the second movement position, the board deflection portion 68A deflects until abutting on the moving portion outer surface 85A of the moving portion 82. At this time, a deflection amount (second deflection amount) δ1b of the board deflection portion 68A becomes less than the deflection amount (first deflection amount) δ1a in the state in which the moving portion 82 is at the first movement position.

As described above, in the present modification, the distance (ζ1) between the board deflection portion 68A and base 17 in the space 67A varies in accordance with the movement of the moving portion 82. Thereby, the deflection amount (δ1) varies in the state in which the board deflection portion 68A has deflected. In addition, the moving portion 82 is moved by the moving operation with the moving operation bar (distance adjusting portion), and the distance (ζ1) between the board deflection portion 68A and base 17 in the space 67A is adjusted by the input of the moving operation. In the meantime, the adjustment of a distance (ζ2) between the board deflection portion 68B and base 17 in the space 67B, and the adjustment of a distance ((ζ3) between the board deflection portion 68C and base 17 in the space 67C, may also be executed by the moving operation bar 81 and moving portion 82, like the adjustment of the distance ((ζ1) between the board deflection portion 68A and base 17 in the space 67A.

Additionally, in the above-described embodiments, etc., the switch board 35 is provided with the three switches 42A to 42C. However, it should suffice if the switch board 35 is provided with at least one switch (42A to 42C). Besides, it should suffice if the pusher (22A to 22C) and the board deflection portion (68A to 68C) are provided in association with each switch (42A to 42C).

Additionally, in the above-described embodiments, the board unit 25 is provided in the inside of the held unit 3 of the energy treatment instrument 2, and the operation input unit 15 including the board unit 25 is provided in the held unit 3. However, the restriction to this is unnecessary. For example, in a certain modification, an imaging device, such as a camera, may be provided with the above-described board unit 25 and operation input unit 15. In this case, the board unit 25 is disposed in the inside of the armor casing of the imaging device.

In the above-described embodiments, etc., the operation input unit (15) includes the board unit (25) which is provided with the switches (42A to 42C); the base (17) on which the board unit (25) is disposed; and the button portion (31A to 31C) which is pushed in an operation input. Each of the external force application portions (32A to 32C) moves along the movement axis (corresponding one of M1 to M3) by the button portion (corresponding one of 31A to 31C) being pushed. The open or closed state of each of the switches (42A to 42C) changes by the external force, which is caused to act on the board unit (25), varying in accordance with the movement of the external force application portion (corresponding one of 32A to 32C). Each of the board deflection portions (68A to 68C) is provided in that region in the board unit (25), where the switch (corresponding one of 42A to 42C) is disposed, and has flexibility. In the state in which the button portion (corresponding one of 31A to 31C) is not pushed, each of the board deflection portions (68A to 68C) forms the space (corresponding one of 67A to 67C) between the board deflection portion and the base (17). In addition, each of the board deflection portions (68A to 68C) deflects toward the movement direction (T2) of the external force application portion (corresponding one of 32A to 32C) by the external force application portion (corresponding one of 32A to 32C) moving by the pushing of the button portion (corresponding one of 31A to 31C).

### Reference Examples

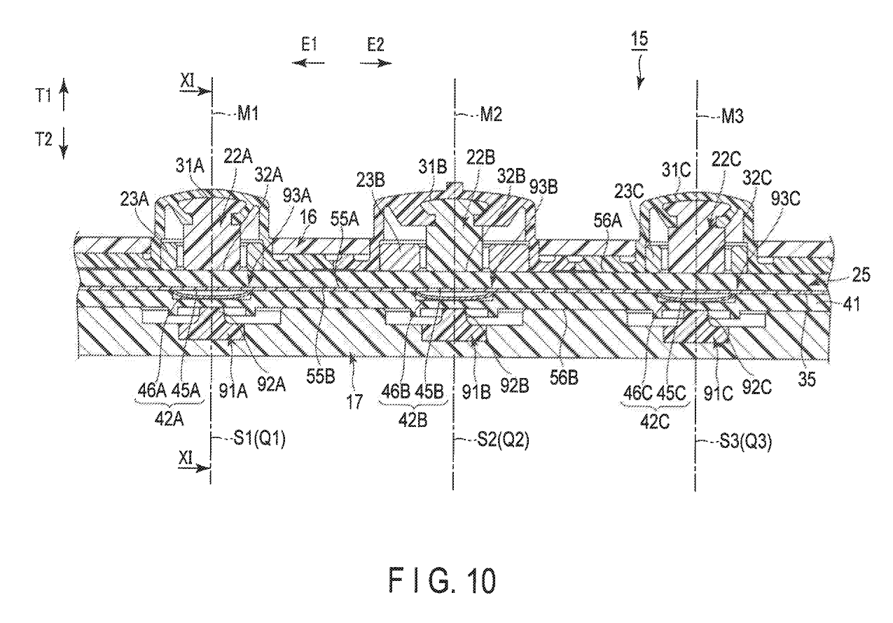

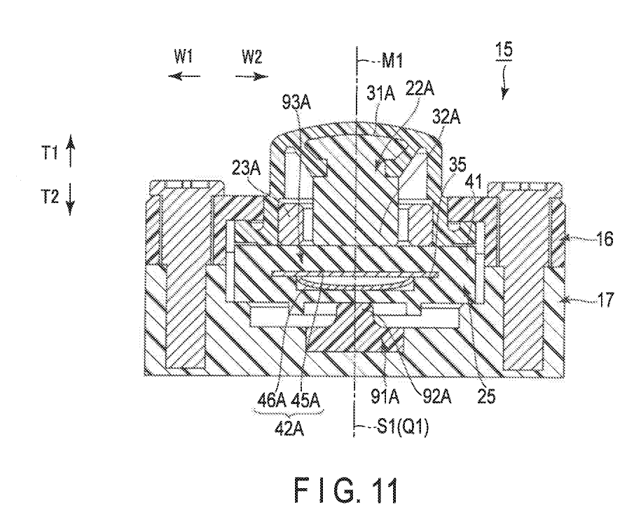

Next, a first reference example will be described with reference to FIG. 10 and FIG. 11. Incidentally, in the first reference example, the same parts as in the first embodiment are denoted by like reference numerals, and a description thereof is omitted.

FIG. 10 and FIG. 11 illustrate an operation input unit 15. FIG. 10 illustrates a cross section perpendicular to the width direction of the energy treatment instrument 2 (the width direction of the board unit 25). In addition, FIG. 11 is a cross-sectional view taken along line XI-XI in FIG. 10. As illustrated in FIG. 10 and FIG. 11, in the present reference example, the board unit 25 is attached to the base 17, in the state in which the cover portion outer surface (second cover portion outer surface) 56B of the cover portion 41 abuts on the installation surface 58 of the base 17. In addition, in the operation input unit 15, reverse pushers 91A to 91C (three reverse pushers in this reference example) are provided. The reverse pushers 91A to 91C are provided in the state in which the reverse pushers 91A to 91C are integral with the base 17 or are fixed to the base 17. Each of the reverse pushers 91A to 91C extend along an extending axis (corresponding one of Q1 to Q3) which is substantially parallel to the thickness direction of the board unit 25. Each of the reverse pushers 91A to 91C includes an external force application portion (corresponding one of 92A to 92C). The external force application portions (second external force application portions) 92A to 92C abut on the cover portion external surface 56B of the cover portion 41 of the board unit 25 from the base 17 side (the arrow T2 side). In addition, each of the reverse pushers 91A to 91C (external force application portions 92A to 92C) is located on the base 17 side with respect to the board unit 25 in the state in which the extending axis (corresponding one of Q1 to Q3) is coaxial (substantially coaxial) with the center axis (corresponding one of S1 to S3) of the corresponding switch (corresponding one of 42A to 42C).

The board unit 25 is provided with board deformation portions 93A to 93C (three board deformation portions in the present embodiment). Each of the board deformation portions 93A to 93C is provided in a region of the board unit 25, where the corresponding switch (corresponding one of 42A to 42C) is located. Thus, each of the board deformation portions 93A to 93C is provided at a position where the center axis (corresponding one of S1 to S3) of the corresponding switch (corresponding one of 42A to 42C), the movement axis (corresponding one of M1 to M3) of the corresponding pusher (corresponding one of 22A to 22C), and the extending axis (corresponding one of Q1 to Q3) of the corresponding reverse pusher (corresponding one of 91A to 91C) pass. In addition, each of the board deformation portions 93A to 93C is formed of a part of the switch board 35 and a part of the cover portion 41. Here, the switch board 35 is a flexible printed board, and the cover portion 41 is formed of an elastic material. Thus, the board deformation portions 93A to 93C have flexibility.

Each of external force application portions (first external force application portions) 32A to 32C abuts on the corresponding board deformation portion (corresponding one of 93A to 93C) from the unit armor portion 16 side (the arrow T1 side). Thus, a first external force by the corresponding first external force application portion (corresponding one of 32A to 32C) acts on each of the board deformation portions 93A to 93C from the unit armor portion 16 side. In addition, each of the external force application portions (second external force application portions) 92A to 92C abuts on the corresponding board deformation portion (corresponding one of 93A to 93C) from the base 17 side (the arrow T2 side). Thus, a second external force by the corresponding second external force application portion (corresponding one of 92A to 92C) acts on each of the board deformation portions 93A to 93C from the base 17 side.

In addition, in the present reference example, the switches 42A to 42C are provided on the board surface (second board surface) 55B which faces the base 17 side in the switch board 35. In addition, unlike the first embodiment, in each of the switches 42A to 42C, the movable contact portion (corresponding one of 46A to 46C) is located on the base 17 side (the arrow T2 side) with respect to the fixed contact portion (corresponding one of 45A to 45C).

In the present reference example, too, in each of the pushers 22A to 22C, the button portion (corresponding one of 31A to 31C) is pushed (i.e. the energy operation is input), and the first external force application portion (corresponding one of 32A to 32C) moves from the neutral state (non-pushed state), and thereby the external force (first external force) acting on the cover portion 41 varies. Each of the board deformation portions 93A to 93C elastically deforms from the non-pushed state of the corresponding button portion (corresponding one of 31A to 31C) in accordance with the variation of the external force (first external force) from the corresponding first external force application portion (corresponding one of 32A to 32C). By the elastic deformation, the abutment state of each of the board deformation portions 93A to 93C upon the second external force application portion (corresponding one of 92A to 92C) varies. Thereby, in each of the board deformation portions 93A to 93C of the board unit 25, the second external force from the base 17 side, which is exerted by the corresponding second external force application portion (corresponding one of 92A to 92C), varies.

In addition, in accordance with the variation of the second external force acting on the corresponding board deformation portion (corresponding one of 93A to 93C) from the corresponding second external force application portion (corresponding one of 92A to 92C), a pushing force acts on each of the switches 42A to 42C from the base 17 side. In each of the switches 42A to 42C, by the pushing force acting from the base 17 side, the movable contact portion (corresponding one of 46A to 46C) is pushed by the cover portion 41, and the movable contact portion (corresponding one of 46A to 46C) elastically deforms into a state of contact with the fixed contact portion (corresponding one of 45A to 45C). Thereby, each of the switches 42A to 42C enters the closed state.

By the movement of the corresponding pusher (corresponding one of 22A to 22C) to the neutral state (non-pushed state), each of the board deformation portions 93A to 93C restores (elastically restores) to the original shape. Thereby, in each of the board deformation portions 93A to 93C, the second external force, which acts from the base 17 side by the corresponding second external force application portion (corresponding one of 92A to 92C), varies. In addition, in accordance with the variation of the external force (second external force) onto the board deformation portion (corresponding one of 93A to 93C) from the corresponding second external force application portion (corresponding one of 92A to 92C), the pushing force no longer acts on each of the switches 42A to 42C from the base 17 side, and the movable contact portion (corresponding one of 46A to 46C) is released from (is no longer in contact with) the fixed contact portion (corresponding one of 45A to 45C). Thereby, each of the switches 42A to 42C enters the open state.

Each of the first external force application portions 32A to 32C (pushers 22A to 22C) is movable along the movement axis (corresponding one of M1 to M3). Hence, a gap of such a degree as to secure mobility of the pusher (corresponding one of 22A to 22C) is formed between each of the pushers 22A to 22C and the corresponding support member (corresponding one of 23A to 23C). Thus, in each of the pushers 22A to 22C, when the first external force application portion (corresponding one of 32A to 32C) has moved by the pushing of the button portion (corresponding one of 31A to 31C), there may be a case in which the movement axis (corresponding one of M1 to M3) of the pusher (corresponding one of 22A to 22C) is deviated from the center axis (corresponding one of S1 to S3) of the corresponding switch (corresponding one of 42A to 42C). Here, consideration is given to a configuration in which the reverse pushers 91A to 91C of the present reference example are not provided. In this configuration, the movement axis (corresponding one of M1 to M3) of the pusher (corresponding one of 22A to 22C) may be deviated from the center axis (corresponding one of S1 to S3) of the switch (corresponding one of 42A to 42C). Consequently, even when the button portion (corresponding one of 31A to 31C) was pushed, there may be a case in which the switch (corresponding one of 42A to 42C) does not properly enter the closed state.

By contrast, in the present reference example, the reverse pushers 91A to 91C (second external force application portions 92A to 92C) are provided in the state in which the reverse pushers 91A to 91C (second external force application portions 92A to 92C) are integral with the base 17 or are fixed to the base 17. Each of the reverse pushers 91A to 91C (second external force application portions 92A to 92C) is located on the base 17 side with respect to the board unit 25, in the state in which the center axis (corresponding one of S1 to S3) of the corresponding switch (corresponding one of 42A to 42C) is coaxial (substantially coaxial) with the extending axis (corresponding one of Q1 to Q3). In addition, the second external force, which is exerted on the board unit 25 from the base 17 side by the corresponding second external force application portion (corresponding one of 92A to 92C), varies, and thereby the open or closed state of each of the switches 42A to 42C changes. Accordingly, each of the switches 42A to 42C enters the closed state by the pushing force from the base 17 side, in the state in which the extending axis (corresponding one of Q1 to Q3) of the corresponding reverse pusher (corresponding one of 91A to 91C) is coaxial (substantially coaxial) with the center axis (corresponding one of S1 to S3). At this time, since the center axis (corresponding one of S1 to S3) of the switch (corresponding one of 42A to 42C) is coaxial (substantially coaxial) with the extending axis (corresponding one of Q1 to Q3) of the corresponding second external force application portion (corresponding one of 92A to 92C), the switch (corresponding one of 42A to 42C) properly enters the closed state. Accordingly, in the present reference example, the open or closed state of each of the switches 42A to 42C can properly be changed over based on the operation input by the corresponding button portion (corresponding one of 31A to 31C).

In the meantime, in a certain reference example, in the configuration in which the reverse pushers 91A to 91C are provided, the board unit 25, which is not provided with the cover portion 41 (i.e. is formed of only the switch board 35), may be disposed on the installation surface 58 of the base 17.

Additionally, in the above-described embodiments, etc., the switch board 35 is provided with the three switches 42A to 42C. However, it should suffice if the switch board 35 is provided with at least one switch (42A to 42C). Besides, it should suffice if the pusher (22A to 22C), reverse pusher (91A to 91C) and the board deformation portion (93A to 93C) are be provided in association with each switch (42A to 42C).

Hereinafter, characteristic items of reference examples are described.

(Item 1)

An operation input unit comprising:

a board unit including a switch;

a base on which the board unit is disposed;

a button portion which is pushed in an operation input;

a first external force application portion configured to move along a movement axis by the button portion being pushed, and configured to vary a first external force, which is caused to act on the board unit from the button portion side in accordance with the movement; and

a second external force application portion provided in a state in which the second external force application portion is integral with the base or is fixed to the base, the second external force application portion being configured to change an open or closed state of the switch by varying the second external force, which is caused to act on the board unit from the base side in accordance with the variation of the first external force from the first external force application portion to the board unit.

(Item 2)

The operation input unit of item 1, wherein the second external force application portion is located on the base side with respect to the board unit in a state in which an extending axis thereof is coaxial with a center axis of the switch.

(Item 3)

The operation input unit of item 1, wherein the board unit includes a board deformation portion configured to elastically deform in accordance with the variation of the first external force from the first external force application portion, whereby an abutment state on the second external force application portion varies, and the second external force acting from the second external force application portion varies.

(Item 4)

The operation input unit of item 1, wherein the switch includes a fixed contact portion and a movable contact portion, the movable contact portion being configured to elastically deform into a state of contact with the fixed contact portion by a pushing force acting from the base side on the switch in accordance with the variation of the second external force acting from the second external force application portion.

(Item 5)

The operation input unit of item 4, wherein the movable contact portion is located on the base side with respect to the fixed contact portion.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An operation input unit comprising:
a board unit including a switch;
a base on which the board unit is disposed;
a button portion which is pushed in an operation input;
an external force application portion configured to move along a movement axis by the button portion being pushed, and configured to change an open or closed state of the switch by varying an external force which is caused to act on the board unit in accordance with the movement;
a board deflection portion provided in a region in the board unit, where the switch is disposed, the board deflection portion having flexibility, being configured to form a space between the board deflection portion and the base in a state in which the button portion is not pushed, and configured to deflect toward a movement direction of the external force application portion by the external force application portion moving by the pushing of the button portion; and
a deflection restriction portion which is configured to restrict a deflection amount of the board deflection portion by the board deflection portion abutting on the deflection restriction portion in a state in which the board deflection portion has deflected toward the movement direction of the external force application portion.

2. (canceled)

3. The operation input unit of claim 1, wherein
the base includes an abutment reception surface on which the board unit abuts, and a recess portion which is recessed from the abutment reception surface toward the movement direction of the external force application portion in a state in which the button portion is pushed, and
the recess portion includes a recess portion bottom surface which is provided with the deflection restriction portion, and which forms the space between the recess portion bottom surface and the board deflection portion in the state in which the button portion is not pushed.

4. The operation input unit of claim 1, wherein the base includes a support portion which supports the board unit in a state in which the support portion abuts on the board unit, and a separate counter-surface which is opposed to the board deflection portion, the separate counter-surface being provided with the deflection restriction portion and forming the space between the separate counter-surface and the board deflection portion in the state in which the button portion is not pushed.

5. The operation input unit of claim 4, wherein the support portion includes an elastic portion which is pushed from the board deflection portion by the board deflection portion deflecting in the movement direction of the external force application portion, and which is configured to elastically contract by the pushing from the board deflection portion.

6. The operation input unit of claim 1, wherein the base includes a base body, and a moving portion which is movable relative to the base body, the moving portion being configured to vary a distance between the board deflection portion and the base in the space in accordance with the movement of the moving portion.

7. The operation input unit of claim 6, further comprising a distance adjusting portion to which an operation of moving the moving portion relative to the base body is input, and which is configured to adjust the distance of the space between the board deflection portion and the base.

8. An energy treatment instrument comprising:
the operation input unit of claim 1; and
a treatment portion configured to be supplied with energy for use in a treatment based on the operation input in the button portion, and configured to perform the treatment by using the supplied energy.
